(19) United States
(12) Patent Application Publication
(10) Pub. No.: US 2021/0284963 A1
(43) Pub. Date: Sep. 16, 2021

(54) METHODS OF OBTAINING A MIXED POPULATION OF HUMAN XCR1+ AND PLASMACYTOID DENDRITIC CELLS FROM HEMATOPOIETIC STEM CELLS

(71) Applicants: INSERM (INSTITUT NATIONAL DE LA SANTÉ ET DE LA RECHERCHE MÉDICALE), Paris (FR); CENTRE NATIONAL DE LA RECHERCHE SCIENTIFIQUE (CNRS), Paris (FR); UNIVERSITÉ D'AIX MARSEILLE, Marseille Cedex 07 (FR)

(72) Inventors: Marc DALOD, Cedex 9 Marseille (FR); Sreekumar BALAN, Cedex 9 Marseille (FR); Catharina ARNOLD-SCHRAUF, Cedex 9 Marseille (FR)

(21) Appl. No.: 16/330,382

(22) PCT Filed: Sep. 4, 2017
(86) PCT No.: PCT/EP2017/072132

§ 371 Date: Mar. 5, 2019

(30) Foreign Application Priority Data
Sep. 5, 2016 (EP) 16306112.0

(51) Int. Cl. C12N 5/0784; A61K 35/15

(52) U.S. Cl. CPC C12N 5/0639; A61K 35/15; C12N 2501/42; C12N 2502/1358; C12N 2501/26; C12N 2501/2307; C12N 2501/145; C12N 2506/11

(57) ABSTRACT

The present invention relates to methods of obtaining a mixed population of human XCR1+ and plasmacytoid dendritic cells from hematopoietic stem cells. Human DC subsets are rare in blood and other tissues, difficult and expensive to isolate, and fragile. Hence, to advance on deciphering their functions and their molecular regulation, there is a strong need for relevant in vitro models. The inventors developed a new protocol allowing simultaneous generation of the various human DC subsets in vitro from hematopoietic progenitors. In particular, the present invention relates to a method of obtaining a mixed population of human XCR1⁺ and plasmacytoid dendritic cells said method comprising the steps of i) culturing a population of hematopoietic stem cells (HSC) or committed hematopoietic precursor cells in the presence of a Notch ligand, and thereafter, ii) isolating human XCR1⁺ and plasmacytoid dendritic cells from the culture.

### FIELD OF THE INVENTION

The present invention relates to methods of obtaining a mixed population of human XCR1⁺ and plasmacytoid dendritic cells from hematopoietic stem cells.

### BACKGROUND OF THE INVENTION

Dendritic cells (DC) are a heterogeneous family of rare leukocytes that sense danger signals and convey them to lymphocytes for the orchestration of adaptive immune defenses.

Clinical trials used monocytes derived DC (MoDC) to attempt to promote protective immunity in patients suffering from infections or cancer. These immunotherapies showed limited efficacy, owing to the poor recirculation of MoDC to lymph nodes (Adema, G J, et al. Migration of dendritic cell based cancer vaccines: in vivo veritas? Curr Opin Immunol. 2005; 17:170-174) (Plantinga, M et. al.. Conventional and Monocyte-Derived CD11b(+) Dendritic Cells Initiate and Maintain T Helper 2 Cell-Mediated Immunity to House Dust Mite Allergen. Immunity. 2013) and likely to other yet uncharacterized functional differences between MoDC and lymphoid tissues-resident DC (LT-DC). Hence, major efforts are being made to better characterize human LT-DC and to evaluate their immunoactivation potential. Steady state human blood and secondary lymphoid organs contain three major DC subsets, CD141(BDCA3)⁺CLEC9A⁺ classical DC (cDC), CD1c(BDCA1)⁺ cDC and CLEC4C(BDCA2)⁺ plasmacytoid DC (pDC) (Ziegler-Heitbrock, L et al. Nomenclature of monocytes and dendritic cells in blood. Blood. 2010; 116:e74-80). Homologies exist between mouse and human LT-DC subsets (Robbins, S H, et al. Novel insights into the relationships between dendritic cell subsets in human and mouse revealed by genome-wide expression profiling. Genome biology. 2008) (Crozat, K, et al. Comparative genomics as a tool to reveal functional equivalences between human and mouse dendritic cell subsets. Immunological reviews. 2010). Comparative transcriptomics (Watchmaker, P B, et al. Comparative transcriptional and functional profiling defines conserved programs of intestinal DC differentiation in humans and mice. Nat Immunol. 2014) (Haniffa, M, et al. Human tissues contain CD141hi cross-presenting dendritic cells with functional homology to mouse CD103+ nonlymphoid dendritic cells. Immunity. 2012; 37:60-73) and functional studies (Crozat, K, et al. The XC chemokine receptor 1 is a conserved selective marker of mammalian cells homologous to mouse CD8alpha+ dendritic cells. J Exp Med. 2010; 207:1283-1292.) (Bachem, A, et al. Superior antigen cross-presentation and XCR1 expression define human CD11c+CD141+ cells as homologues of mouse CD8+ dendritic cells. J Exp Med. 2010; 207:1273-1281) (Jongbloed, S L et al. Human CD141+ (BDCA-3)+ dendritic cells (DCs) represent a unique myeloid DC subset that cross-presents necrotic cell antigens. J Exp Med. 2010; 207:1247-1260) showed that human CD141⁺CLEC9A⁺ cDC are homologous to mouse spleen CD8α⁺ cDC, which are specialized in cross-presentation. Mouse CD8α⁺ cDC and human CD141⁺CLEC9A⁺ cDC specifically express the XCR1 chemokine receptor (Dorner, B G et al. Selective expression of the chemokine receptor XCR1 on cross-presenting dendritic cells determines cooperation with CD8+ T cells. Immunity. 2009; 31:823-833) (Crozat, K, et al. Cutting edge: expression of XCR1 defines mouse lymphoid-tissue resident and migratory dendritic cells of the CD8alpha+ type. J Immunol. 2011; 187:4411-4415) and can therefore be coined XCR1⁺ cDC. The ligands of XCR1 are selectively expressed in Natural Killer (NK) and CD8 T cells, promoting their interactions with XCR1⁺ cDC. Human XCR1⁺ cDC have been described in many tissues (Yoshio, S et al. Human blood dendritic cell antigen 3 (BDCA3)(+) dendritic cells are a potent producer of interferon-lambda in response to hepatitis C virus. Hepatology. 2013; 57:1705-1715). Human and mouse XCR1⁺ cDC specifically express high levels of Toll-like receptor (TLR)-3 (Crozat, K, Vivier, E, Dalod, M. Crosstalk between components of the innate immune system: promoting anti-microbial defenses and avoiding immunopathologies. Immunological reviews. 2009; 227:129-149) and respond to its triggering with hepatitis C virus or with the synthetic ligand polyinosinic-polycytidylic Acid (PolyL-C) by interferon (IFN)-λ production (Zhang, S et al. Human type 2 myeloid dendritic cells produce interferon-lambda and amplify interferon-alpha in response to hepatitis C virus infection. Gastroenterology. 2013; 144:414-425 e417) and by enhanced cross-presentation. The extent to which human XCR1⁺ cDC are more efficient for cross-presentation than other human DC subsets is debated. It depends on the tissue origin of the DC subsets, on their activation status and on the mode of antigen delivery (Segura, E et al. Similar antigen cross-presentation capacity and phagocytic functions in all freshly isolated human lymphoid organ-resident dendritic cells. J Exp Med. 2013; 210:1035-1047) (Cohn, L, et al. Antigen delivery to early endosomes eliminates the superiority of human blood BDCA3+ dendritic cells at cross presentation. J Exp Med. 2013; 210:1049-1063) (Flinsenberg, T W, et al. Fcgamma receptor antigen targeting potentiates cross-presentation by human blood and lymphoid tissue BDCA-3+ dendritic cells. Blood. 2012; 120:5163-5172). However, several independent studies showed that human XCR1⁺ blood cDC (bcDC) excel at cross-presentation of cell-associated antigens and of particulate antigens delivered through Fcγ receptors, through lysosomes or upon PolyL-C stimulation (Nizzoli, G et al. Human CD1c+ dendritic cells secrete high levels of IL-12 and potently prime cytotoxic T cell responses. Blood. 2013). Since they share unique characteristics with mouse XCR1⁺ cDC, human XCR1⁺ bcDC constitute a distinct human DC subset that may have potential clinical applications (Gallois, A, Bhardwaj, N. A needle in the ‘cancer vaccine’ haystack. Nat Med. 2010; 16:854-856) (Radford, K J, Caminschi, I. New generation of dendritic cell vaccines. Hum Vaccin Immunother. 2013; 9) (Tacken, P J, Figdor, C G. Targeted antigen delivery and activation of dendritic cells in vivo: steps towards cost effective vaccines. Semin Immunol. 2011; 23:12-20). Accordingly there is a need for having in vitro method of obtaining such cells. Recently, the inventors described a protocol for the in vitro generation of human XCR1⁺ cDC from CD34⁺ hematopoietic progenitors (Balan S, Dalod M. In Vitro Generation of Human XCR1(+) Dendritic Cells from CD34(+) Hematopoietic Progenitors. Methods Mol Biol. 2016; 1423:19-37. doi: 10.1007/978-1-4939-3606-9_2). Immunotherapy with autologous human pDC directly isolated ex vivo, loaded in vitro with antigens and matured upon exposure to an attenuated virus vaccine, did recently yield promising results in melanoma patients (Tel J, et al. Natural human plasmacytoid dendritic cells induce antigen-specific T-cell responses in melanoma patients. Cancer Res. 2013; 73:1063-75). In mice, cross-talk between pDC and XCR1⁺ cDC can be critical for the induction of optimal, protective, adaptive immunity to viral infections and also to cancer (Nierkens S, et al. Immune adjuvant efficacy of CpG oligonucleotide in cancer treatment is founded specifically upon TLR9 function in plasmacytoid dendritic cells. Cancer Res. 2011; 71:6428-37) (Zhang Y, et al. Genetic vaccines to potentiate the effective CD103+ dendritic cell-mediated cross-priming of antitumor immunity. J Immunol. 2015; 194:5937-47). Recent correlative data in a human clinical trial does support a protective role of the cross-talk between pDC and XCR1⁺ cDC for cancer immunotherapy (Sluijter B J, et al. Arming the Melanoma Sentinel Lymph Node through Local Administration of CpG-B and GM-CSF: Recruitment and Activation of BDCA3/CD141(+) Dendritic Cells and Enhanced Cross-Presentation. Cancer Immunol Res. 2015; 3:495-505). The rarity and fragility of human XCR1⁺ cDC is a major limitation to their direct isolation ex vivo for immunotherapy. Hence, methods of obtaining a mixed population of human XCR1⁺ cDC and pDC from hematopoietic stem cells are of strong interest to advance our basic understanding of their interactions and as a potential source of cells for immunotherapy. A few studies have reported simultaneous in vitro generation of human XCR1+ cDC and pDC from hematopoietic stem cells but with limited yields (Thordardottir et al. Stem cells and development. 2014; Lee et al. J Exp Med. 2015).

### SUMMARY OF THE INVENTION

The present invention relates to methods of obtaining a mixed population of human XCR1⁺ and plasmacytoid dendritic cells from hematopoietic stem cells, leading to higher yields than reported previously and including an expansion phase of the precursors before their differentiation making this culture system highly versatile. In particular, the present invention is defined by the claims.

### DETAILED DESCRIPTION OF THE INVENTION

The present invention relates also to a method of obtaining a mixed population of human XCR1+ and plasmacytoid dendritic cells comprising the steps of i) culturing a population of human hematopoietic stem cells (HSC) or committed hematopoietic precursor cells in the presence of a Notch ligand, and thereafter, ii) isolating human XCR1+ and plasmacytoid dendritic cells from the culture.

As used herein, the term “classical dendritic cell” or “cDC” has its general meaning in the art and refers to a population of hematopoietic cells with critical roles in immunity, including immune activation in response to pathogen-elicited danger signals and immune tolerance. These cells are characterized by their distinctive morphology and high levels of surface MHC-class II expression. cDC have a high capacity for sensitizing MHC-restricted T cells, and are the only antigen-presenting cells (APCs) that can efficiently activate naïve T-cells.

As used herein, the term “XCR1” has its general meaning in the art and refers to the XC chemokine receptor 1. An exemplary human amino acid sequence is represented by the NCBI reference sequence NP_001019815.1. XCR1 is also known as GPRS; CCXCR1.

As used herein, the term “XCR1⁺ cDC” has its general meaning in the art and refers to a subset of dendritic cells that specifically express the XCR1 chemokine receptor. Human XCR1⁺ cDC are particularly efficient for cross-presentation. As components of the innate immune system, these cells express intracellular Toll-like receptors 3 and 8, which enable the detection of viral nucleic acids, such as dsRNA and ssRNA motifs respectively. Upon stimulation and subsequent activation through TLR3, these cells uniquely produce large amounts of Type III interferon (e.g., IFN-λ), which are critical pleiotropic anti-viral compounds mediating a wide range of effects. Upon stimulation and subsequent activation through TLR8, these cells can produce interleukin-12 (IL-12), which is a critical cytokine contributing to promote functional polarization of T lymphocytes towards potent antiviral and anti-tumoral functions.

As used herein, the term “plasmacytoid dendritic cell” or “pDC” has its general meaning in the art and refers to a subtype of circulating dendritic cells found in the blood and peripheral lymphoid organs. These cells express the surface markers CD123, BDCA-2(CD303), BDCA-4(CD304) and HLA-DR, but do not express CD11c, CD14, CD3, CD20 or CD56, which distinguishes them from cDC, monocytes, T-cells, B cells and NK cells. As components of the innate immune system, these cells express intracellular Toll-like receptors 7 and 9, which enable the detection of viral and bacterial nucleic acids, such as ssRNA or CpG DNA motifs. Upon stimulation and subsequent activation, these cells produce large amounts of Type I interferon (mainly IFN-α and IFN-β) and Type III interferon (e.g., IFN-λ), which are critical pleiotropic anti-viral compounds mediating a wide range of effects.

As used herein, the term “hematopoietic stem cell” or “HSC” has its general meaning in the art and refers to immature blood precursor cells having the capacity to self-renew and to differentiate into more mature blood cells comprising granulocytes (e.g., promyelocytes, neutrophils, eosinophils, basophils), erythrocytes (e.g., reticulocytes, erythrocytes), thrombocytes (e.g., megakaryoblasts, platelet producing megakaryocytes, platelets), monocytes (e.g., monocytes, macrophages), lymphocytes (e.g. B- and T cells), and DC. In particular, hematopoietic stem cell are CD34⁺ cells. The term “CD34⁺ cells” refers to cells that express at their surface the CD34 marker. Hematopoietic stem cells and in particular CD34⁺ cells are typically obtained from blood products. A blood product includes a product obtained from the body or an organ of the body containing cells of hematopoietic origin. Such sources include un-fractionated bone marrow, umbilical cord blood, peripheral blood, liver, thymus, lymph and spleen. All of the aforementioned crude or un-fractionated blood products can be enriched for cells having hematopoietic stem cell characteristics in ways known to those of skill in the art.

As used herein, the term “committed precursor cells” refers to cells which develop from HSC or CD34⁺ cells but have a more restricted developmental potential. Consequently, these precursor cells (e.g. macrophage dendritic cell precursor, common dendritic cell precursor, or pre-dendritic cell precursor) are more committed to develop into a particular immune cell lineage (e.g macrophages, DC).

In some embodiments, the method of the present invention involves culturing of human CD34⁺ cells that have been isolated, or partially purified, from cord blood. CD34⁺ cells may be isolated from cord blood using any of the methods well known to persons skilled in the art. One preferred method involves the isolation of CD34⁺ cells from the fraction(s) of centrifuged cord blood which remain following removal of erythrocytes, by magnetic bead-based methods such as the magnetically activated cell sorting (MACS) protocol described in the CD34 MicroBead Kit from Miltenyi Biotec (Miltenyi Biotec GmbH, Cologne, Germany (2006)).

In some embodiments, the population of CD34⁺ cells was previously expanded in an appropriate culture medium before being cultured in the presence of the Notch ligand. The term “expansion” refers to growing cells in culture to achieve a larger population of the cells.

As used herein, the term “Notch ligand” has its general meaning in the art and refers to a protein or peptide that binds to a Notch receptor and activates a Notch signaling pathway. The Notch ligand used in the present invention can be derived from any mammalian species, and includes human and non-human Notch ligands. Preferably, the Notch ligand is capable of activating a human notch receptor, including Notch1, Notch2, Notch3, Notch4, or any combination thereof. Notch ligands include Delta-like-ligands (DLL) and Jagged ligands.

In some embodiments, the Notch ligand is Delta1 (Delta-like 1/DLL1) or Delta4 (Delta-like 4/DLL4).

In some embodiments, the Notch ligand is immobilized on a solid phase. In some embodiments, the solid phase is the surface of a tissue culture dish, flask, or a bead.

In some embodiments, the Notch ligand is provided to the culture medium by the inclusion of suitable feeder cells. As used herein, the term “feeder cell” is a cell that grows in vitro, that is co-cultured with another population of cells (e.g. HSC). Accordingly, in some embodiments, step i) consists of co-culturing the hematopoietic stem cells with the feeder cells. Suitable feeder cells may include foetal liver stromal feeder cells such as AFT024 (Moore, K. A. et al., 1997), and bone marrow stromal feeder cells such as L87/4 and L88/5 (Thalmeier, K. et al. 1994), AC6.21 (Shih, C C. et al, 1999), MS5 (Lee J, Breton G, Aljoufi A, Zhou Y J, Puhr S, Nussenzweig M C, Liu K. Clonal analysis of human dendritic cell progenitor using a stromal cell culture. J Immunol Methods. 2015 October; 425:21-6. doi: 10.1016/j.jim.2015.06.004.) and FBMD-I (Kusadasi, N. et al., 2000), which are well known to persons skilled in the art. Typically, the feeder cell is an OP9 bone marrow stromal feeder cell (i.e. ATCC CRL-2749™) which has been transformed with, and stably expresses, an exogenous nucleic acid molecule encoding the Notch Ligand such as DLL1. In some embodiments, the feeder cells are OP9-DLL1 feeder cells that are commercially available. In some embodiments, the hematopoietic stem cells are co-cultured with a mixture of feeder cell that express the Notch ligand and feeder cells that do not express the Notch ligand. In some embodiments, the hematopoietic stem cells are co-cultured with a mixture of OP9 and OP9-DLL1 cells. Typically the mixture comprises at least 15; 16; 17; 18; 19; 20; 21; 22; 23; 24; 25; 26; 27; 28; 29; 30; 31; 32; 33; 34; 35; 36; 37; 38; 39; 40; 41; 42; 43; 44; 45; 46; 47; 48; 49; or 50% of OP9 cells.

Typically, the feeder cells are adherent cells and are cultured in appropriate culture system such as plates or dishes, so that the feeder cells form a layer. Culture conditions may vary, but standard tissue culture conditions form the basis of the co-culture. Typically, cells are incubated in 5% CO2 incubators at 37° C. in a culture medium.

As used herein, the term “culture medium”, refers to a chemical composition that supports the growth and/or differentiation of a cell, suitably of a mammalian cell. Typical culture media include suitable nutrients (e.g. sugars, amino acids, proteins, and the like) to support the growth and/or differentiation of a cell. Media for the culture of mammalian cells are well known to those of skill in the art and include, but are not limited to Medium 199, Eagle's Basal Medium (BME), Eagle's Minimum Essential Medium (MEM), alpha modification MEM (MEM), Minimum Essential Medium with Non-Essential Amino Acids (MEM/NEAA), Dulbecco's Modification of Eagle's Medium (DMEM), McCoy's 5 A, Rosewell Park Memorial Institute (RPMI) 1640, modified McCoy's 5 A, Ham's F10 and F 12, CMRL 1066 and CMRL 1969, Fisher's medium, Glasgow Minimum Essential Medium (GMEM), Iscove's Modified Dulbecco's Medium (IMDM), Leibovitz's L-15 Medium, McCoy's 5A medium, S-MEM, NCTC-109, NCTC-135, Waymouth's MB 752/1 medium, Williams' Medium E, and the like.

In some embodiments, the culture medium comprises an amount of at least one human cytokine that is suitable for enhancing the dendritic cell differentiation or expansion that occurs during the step of culturing to thereby increase the relative amount of XCR1⁺ cDC. In some embodiments, the human cytokine is selected from the group consisting of FLT-3L, IL-7 and TPO. As used herein, the term ‘FLT-3L’ has its general meaning in the art and refers to Fms-like tyrosine kinase 3 ligand. As used herein, the term “IL-7” has its general meaning in the art and refers to the interleukin 7. As used herein, the term “TPO” has its general meaning in the art and refers to thrombopoietin. In some embodiments, the culture medium comprises an amount of FLT-3L, IL-7 and TPO. The cytokine is provided in the culture medium at a concentration in the range of 1-50 ng/ml. In some embodiments, the culture medium comprises 15 ng/ml of FLT3-L, 7.5 ng/ml of IL-7 and 2.5 ng/ml of TPO.

Typically, the duration of the culturing step is in the range of about 5 to 25 days, more preferably about 14 to 21 days (2-3 weeks). In some embodiments, the duration of the culturing step is 14, 15, 16, 17, 18, 19, 20 or 21 days.

The step of isolating XCR1⁺ and plasmacytoid DC from the culture may be conducted in accordance with any of the methods well known to persons skilled in the art, for example magnetic bead-based methods and FACS cell sorting techniques. For FACS cell sorting, the sorting or “gating” may preferably be conducted in a manner so as to isolate those cells present in the culture which show the appropriate surface marker phenotype. Typically, the CD123(neg) cells in the culture encompass BDCA3(high) cells and the fraction of those that is positive for CLEC9A and CADM1 represents the XCR1⁺ cDC in the culture. The CD123⁺ cells in the culture encompass BDCA2⁺ cells which represent the plasmacytoid DC in the culture.

The method of the present invention is particularly suitable for the preparation of large amounts of DC which can be subsequently used e.g. for research or therapeutics applications.

In particular, the method of the present invention is particular suitable for the preparation of DC vaccine. Thus, another aspect of the present invention relates to a method for the preparation of a DC vaccine comprising the method of the present invention.

As used herein the term “DC vaccine” refers to a vaccine comprising a therapeutically effective amount of DC loaded with an antigen. In some embodiments, the DC are autologous. As used herein the term “autologous” means that the donor and recipient of DC is the same subject. The DC vaccines of the present are particular suitable for the treatment of infectious diseases, cancer or auto-immune diseases.

As used herein, the term “antigen” refers to any molecule or molecular fragment that, when introduced into the body, induces a specific immune response (i.e. humoral or cellular) by the immune system. Antigens have the ability to be bound at the antigen-binding site of an antibody. Antigens are usually proteins or polysaccharides. As used herein, the term “antigen-loaded DC refers to DC that have captured an antigen and processed it for presentation to CD4 T helper cells and CD8 cytotoxic T lymphocytes in association with HLA-class II and HLA-class I molecules, respectively. In some embodiments, the antigen is a viral, a bacterial, a fungal or a protozoal antigen. In some embodiments, the antigen is a tumor-associated antigen (TAA). In some embodiments, the antigen is an auto-antigen. In some embodiments, the antigen is an allergen. In some embodiments, the antigens are molecules that are exogenously administered for therapeutic or other purposes and may trigger an unwanted immune response (e.g. therapeutic clotting factor VIII in haemophilia A or factor IX in haemophilia B).

The invention will be further illustrated by the following figures and examples. However, these examples and figures should not be interpreted in any way as limiting the scope of the present invention.

### FIGURES

FIG. 1. pDC and XCR1⁺ cDC can be efficiently generated from human CD34⁺ cord blood cells. Whereas OP9 cells preferentially support pDC development, OP9_DLL1 cells enhance XCR1⁺ cDC development. A combined feeder layer composed of OP9+OP9_DLL1 cells allows the efficient differentiation of both pDC and XCR1⁺ cDC. (A) General scheme of the culture protocol. CD34⁺ cord blood cells were expanded for 7 days in the presence of FLT3-L, IL-7, TPO, and SCF in a 96 round bottom plate. On day 7 cells were harvested, counted and adjusted to 10,000 cells/ml and transferred onto OP-9, OP9_DLL1, or OP9+OP9_DLL1 feeder layer cells seeded 24 h before in a 24 well flat bottom plate. Cells were differentiated in the presence of FLT3-L, IL-7, and TPO for 14 to 21 days with medium changes every 7 days. Alternatively, expanded cells were frozen on day 7 after expansion for later use. (B) On day 21 of differentiation, cells were harvested and characterized by flow cytometry. pDC were identified as CD206(neg) CD14(neg) CD123(pos) BDCA2(pos) cells. XCR1⁺ cDC were identified as CD206(neg) CD14(neg) CLEC9A(pos)⁺ and CADM1(pos)⁺ oor BDCA3(pos) cells. Plots show one representative donor (CB204) differentiated on the 3 different feeder layer cells in the same experiment. The circle on the right depicts the percent of pDC and XCR1+ cDC in each culture condition. Data are representative of 6 donors. (C) Frequencies of XCR1⁺ DC (top) and pDC (bottom) among total live cells on day 18-21 after differentiation on the 3 different feeder layer cells. (D) Absolute numbers of XCR1⁺ cDC (top) and pDC (bottom) obtained on day 18-21 after differentiation on the 3 different feeder layer cells upon differentiation of 10E4 progenitors expanded from CB CD34+ cells. For C and D, graph shows pooled results from 6 donors. Each donor cells were grown on the 3 different feeder layers in parallel in the same experiment. Statistics were performed using the Wilcoxon matched-pairs signed rank test. *,p<0.05; ns, not significant.

FIG. 2. Notch signaling promotes the development of XCR1⁺ cDC from human CD34⁺ cord blood cells. (A) Scheme of the experimental design. Expanded CD34⁺ cord blood cells were differentiated on OP9_DLL1 feeder layer cells in the presence or absence of the γ-secretase inhibitor DAPT or its vehicle DMSO added on day 0, 7 and 14. (B) The frequency and number of pDC and XCR1⁺ cDC in the cell cultures were assessed by flow cytometry on day 18-21 of differentiation as depicted for FIG. 1B. (C-D) Frequencies (C) and absolute numbers (D) of XCR1⁺ cDC (top) and pDC (bottom) among total live cells. Pooled data from 8 donors are depicted. Statistics were performed using the Wilcoxon matched-pairs signed rank test.*, p<0.05; **, p<0.01; ns, not significant.

FIG. 3. Notch signaling is required early during the differentiation phase of the culture protocol for the promotion of the development of XCR1⁺ cDC. (A) Table displaying the experimental set-up for kinetic analysis of DAPT effect. Medium (untreated), the γ-secretase inhibitor DAPT, or DMSO was added on one or several days during differentiation (day 0, 7, 14) to define in which time frame DAPT acts to inhibit XCR1⁺ cDC development. (B) The frequency of XCR1⁺ cDC (left) and pDC (right) among total live cells after DMSO or DAPT treatment at the indicated time points. Data from one representative donors out of 3 are depicted, with 3 replicate wells per condition. (C) Total numbers of live XCR1⁺ cDC (left) and pDC (right) after DMSO or DAPT treatment at the indicated time points. Numbers are normalized to DMSO. Pooled data from 3 donors are shown. Dots represent mean values for individual triplicate wells for each condition for each donor.

FIG. 4. In vitro derived XCR1⁺ cDC and pDC harbor responses to TLR triggering similar to those of their in vivo counterparts. At the end of the differentiation phase, cultures were stimulated for 6 h or 16 h with ligands for TLR3 (PolyI:C), TLR4 (LPS), TLR7/8 (R848) or TLR9 (CpG2216), with addition of brefeldin A during the last two hours to prevent cytokine secretion. Cells were then cell surface stained for expression of the maturation marker HLA-DR, CD80, CD83 and CD86 (A) or, after fixation and permeabilization, intracellularly stained for the cytokines IFN-α and IFN-λ (B) or IL-12 and TNF (C). The data shown are from one culture representative of independent ones.

### EXAMPLE

Materials

Cell Lines and Feeder Layer Preparation
1 OP9, OP9-DLL1
2 α-MEM glutamax (32561-029—Life technologies).
3 T75 mL flask
4 24 well plates
5 Medium 1: α-MEM glutamax, 20% FCS, 10 mM HEPES, 1 mM sodium pyruvate, Penicillin, Streptomycin, 2 mM L-Glutamin, 50 μM β mercaptoethanol, NEAA

Expansion of Hematopoietic Precursors
1 α-MEM glutamax.
2. FCS
3. Recombinant human cytokines: FLT3-L, SCF, IL-7, TPO (Peprotech)
4. Amplification medium: α-MEMglutamax, FCS 10%, FLT3-L (25 ng/ml), SCF (2.5 ng/ml), IL-7 (5 ng/ml) and TPO (5 ng/ml), to be prepared extemporaneously
5. U-bottom 96-well tissue-culture-treated plates

Cryopreservation and Revival of Expanded Hematopoietic Precursors
1 Iscove's modified delbecoves medium (IMDM)
2 DMSO
3 Deoxyribonuclease I from bovine pancreas (Nalgene, Sigma Aldrich)
4 FCS
5 Cryotubes, e.g. Nunc® CryoTubes®, cryogenic vial, 1.8 ml, internal thread, round bottom, starfoot, free standing (Sigma)
6 Isopropanol
7 Freezing Container (e.g. Mr. Frosty, Nalgene)
8 Freezing medium #1 (FM1): IMDM, 30% FCS
9 Freezing medium #2 (FM2): IMDM, 30% FCS, 20% DMSO, to be prepared extemporaneously
10 15 ml or 50 ml polypropylene tissue culture falcon tube
11 Waterbath adjustable to 37° C.

Differentiation of DC from Expanded Hematopoietic Precursors
1 α-MEM glutamax
2 FCS
3 Recombinant human cytokines: FLT3-L, TPO, IL-7 (Peprotech)
4 Medium #2: α-MEM glutamax, 10% FCS, 10 mM HEPES, 1 mM sodium pyruvate, Penicillin, Streptomycin, 2 mM L-Glutamin, 50 μM β mercaptoethanol, NEAA
5 Differentiation medium #1: Medium #2, 15 ng/ml FLT3-L, 5 ng/ml IL-7 and 2.5 ng/ml TPO, to be prepared extemporaneously.
6 Differentiation medium #2: Medium #2, 30 ng/ml FLT3-L, 10 ng/ml IL-7 and 5 ng/ml TPO, to be prepared extemporaneously.

7 24-well tissue culture-treated plates
8 15 ml or 50 ml polypropylene tissue culture falcon tube

Staining for Flow Cytometry Analysis
1 Fluorochrome-coupled monoclonal antibodies depending on the intended cell populations or biological process to study. The important antibodies are CD206, CD14, BDCA2, CD123, BDCA3, CLEC9A, CADM1, ILT7 etc.
2 U-bottom 96well tissue culture-treated plates
3 FACS buffer: PBS, 1 mM EDTA, 10 mM HEPES
4 Staining buffer (SB): FACS buffer, 2% FCS
5 Human TruStain FcX™ (Fc Receptor Blocking Solution, Biolegend)
6 Blocking buffer (BB): SB complemented 1:20, vol:vol, with Human TruStain FcX™ (e.g. 50 μl TruStain FcX™ for 1 ml SB)
7 LIVE/DEAD® Fixable Aqua Dead Cell Stain Kit (Invitrogen)
8 0.5% paraformaldehyde working solution: prepare 4% (w/v) stock solution in PBS, adjusted to pH7, according to manufacturer's instructions. Stock solution should be aliquoted in 10 ml volumes in 15 ml polypropylene tubes and frozen at −20° C. Extemporaneously prepare 0.5% working solution by diluting stock solution 1/8 in PBS.
9 OneComp eBeads (eBioscience) for compensation control
10 Fluorescence-activated cell sorter for analysis of cells

Methods

The culture system uses the adherent cell lines OP9 or OP9+OP9-DLL1 as the feeder layer for the differentiation of CB_CD34⁺ cells. CD34⁺ cells can differentiated to different DC subsets with or without the 7 day amplification step. The amplification step allows the large scale proliferation of the cells and increases the total number of pDC or XCR1⁺ DC generated from unit number of CD34⁺ cells. This procedure is also helpful for the cryopreservation of the amplified precursors as well as the gene inactivation strategies via shRNA-mediated knock-down or CRISPR/Cas9-mediated knock-out.

Maintenance of the Cells Lines and Preparation of the Feeder Layers
1 OP9 or OP9-DLL1 cell line are maintained with medium 1 (α-MEM glutamax+20% FCS+Supplements) and the cell lines are passaged in each 48-72 hrs, when they are 80-90% confluent. Cells lines can be maintained in T75 or T25 flasks.
2 Adherent cells are detached using 0.05% Trypsin EDTA. Bring the Trypsin EDTA to 37° C. by incubating in water bath set at 37° C.
3 Remove the spent medium gently with pipettes and incubate the cells for 1.5-2 minutes with warm trypsin (0.05%). Use 3-4 mL Trypsin for T25 and 6-7 mL for T75 flask.
4 After 1.5-2 min gently remove the trypsin leaving 1 mL and gently tap the flask for dissociating the cells.
5 Collect the cell by adding 5 mL of fresh medium and the centrifuge the tube at 1500 RPM for 5 minute and suspend the cell pellets in fresh 5 ml medium.
6 Add 1-1.5 mL of cell suspension to new T75 flask and make up the volume to 15 mL (2-3×10⁵ cells).

Expansion of Hematopoietic Precursors
1 Prepare the Amplification medium as described in the material section.
2 Wash the CD34⁺ cells and resuspend them in Amplification medium (α-MEM+10% FCS+Cytokines) at a cell density of 2.5×10⁴ CD34⁺ cells/ml.
3 Plate 200 μL/well of the cell suspension, in U-bottom 96-well tissue culture-treated plates.
4 Harvest the cells on 7^{th} day: transfer the cells into 15 ml or 50 ml tubes and centrifuge at 450 g for 5 minutes.
5 Resuspend the cells in α-MEM glutamax+10% FCS and determine the viable cell count using trypan blue.
6 Expanded cells can be either directly used for setting up the differentiation culture or cryopreserved for future use.

Cryopreservation of Expanded Hematopoietic Precursors
1 The day before, prepare the freezing container by replenishing with fresh isopropanol according to the manufacturer's instructions. Pre-cool it overnight at around +4° C.
2 Prepare FM1 and FM2 and incubate them on ice for a time long enough to allow them to cool to +4° C. (for >=10 min, depending on the volume).
3 Label the appropriate number of cryotubes with sample name, cell number, date etc.
4 Cool the cryotubes in ice for >10 min.
5 Harvest the cell culture and determine the viable count.
6 Re-suspend the cells in FM1, in half of the final volume of cell suspension to be frozen.
7 Keep the cell suspension in ice for a time long enough to allow it to cool to +4° C.
8 Add drop by drop to the cell suspension an identical volume of FM2, to achieve a 1:1 mixture of cell suspension and FM2, with continuous gentle agitation of the cell suspension tube. The tubes must be kept cold, on ice, during the entire procedure.
9 Transfer the cells to cryotubes, on ice.
10 Transfer the vials to the pre-cooled freezing container.
11 Cool the freezing container at −80° C. overnight.
12 The day after, transfer the vials to liquid nitrogen for long term storage.

Revival of Frozen Expanded Hematopoietic Precursors
1 Set the water bath at 37° C.
2 Transfer the vials to the water and thaw the cells rapidly until only a small piece of ice is left in the tube.
3 Transfer the cells to a 15 ml polypropylene tissue culture tube.
4 Dilute the cell suspension 5-fold in cold IMDM, 5% FCS, 20 U/ml DNase I.
5 Gently mix the cell suspension, on ice.
6 Centrifuge the cell at 450 g for 5 minute at low break.
7 Resuspend the cells in Medium #2.

Preparation of the Feeder Layer for CD34⁺ Cell Co-Culture
1 Harvest the OP9 cell lines 48 hrs after seeding (80% confluent) as described above.
2 Dispense 12,500 cells/well in 24 well plate and make the final volume to 500 μL with medium #1. For the co-culture of OP9 and OP9_DLL1, mix cells at a ratio of 75% (OP9) to 25% (OP9_DLL1) and plate them at 12,500 cells/well as described before. Keep the plates for 24 hrs in incubator (see the Notes section).

Co Culture:
1 CD34⁺ cell or 7 days expanded CD34⁺ cells can be used for the co-culture. These cells are seeded on the feeder layer prepared with OP9 or OP9+OP9_DLL1 one day in advance and cultured with the cytokine cocktail for 2-3 weeks. The feeder layer in 24 well plate should be uniformly distributed and covering at least 80-90% of the surface area before the co-culture.
2 Remove the 500 μL medium from each well without disturbing the feeder layer.
3 Distribute the 10⁴ cells/well and add the cytokines (FLT3-L—15 ng/mL, IL-7 5 ng/mL, TPO 2.5 ng/mL make up the final volume to 1 ml with culture medium (Medium #1—α-MEM glutamax+10% FCS+Supplements)
4 On day 7 gently remove 500 μL of medium without disturbing the feeder layer and cells.
5 Carefully add 500 μL of medium #2 (α-MEM glutamax+10% FCS+Supplements+2× cytokines). This step is very critical and should be done carefully and gently; otherwise the feeder layer can detach which will affect DC differentiation.
6 Cells can be harvested on day 14 or maintain for another 7 days (21 days) with the procedure described in step 4.
7 Harvest the cells including the feeder layer by mixing with pipette and collect the cells from all the wells in 15 mL or 50 mL tubes.
8 Gently mix the cell suspension with a 5 mL pipette to make a single cell suspension and detach the DC from feeder layer.
9 Transfer the cell suspension through a 70 μM strainer or muslin cloth to a new 15 mL or 50 mL tube to remove the cell clumps.
10 Centrifuge the tubes at 1500 RPM for 5 minutes and suspend in fresh α-MEM glutamax+10% FCS and determine the viable count using trypan blue.

Phenotypic Identification of the Different Cell Populations at the End of the Culture

The cultures encompasses three different populations based on the expression of CD206 and CD14: CD206⁺CD14^{+/-}, CD206⁻CD14⁺ and CD206⁻CD14⁻ cells. The CD206⁻CD14⁻ fraction encompass a CD123^{high} fraction positive for BDCA2 that represents the pDC in the culture. The CD123^{neg} cells in the culture encompass BDCA3^{high} cells, and the fraction of those that is positive for CLEC9A and CADM1 represents the XCR1⁺ cDC in the culture.

Results

A Mixture of OP9 and OP9_DLL1 Leads to High Yields of Both pDC and XCR1⁺ cDC.

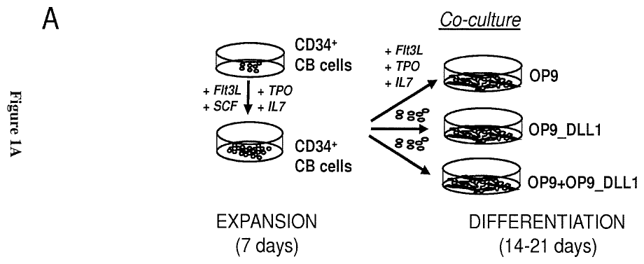

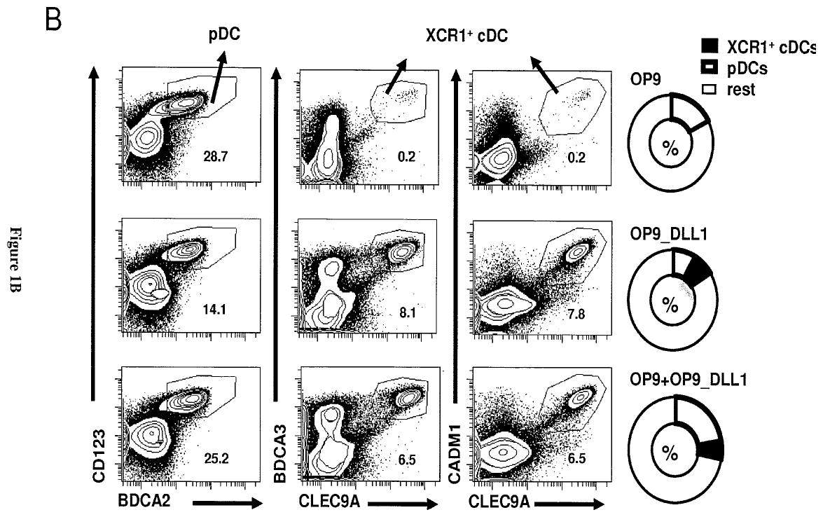

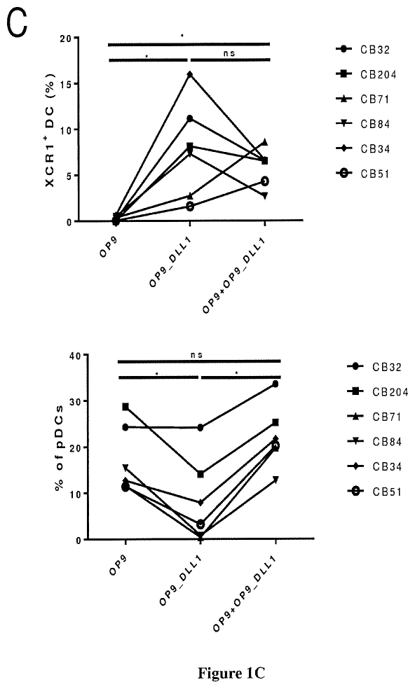

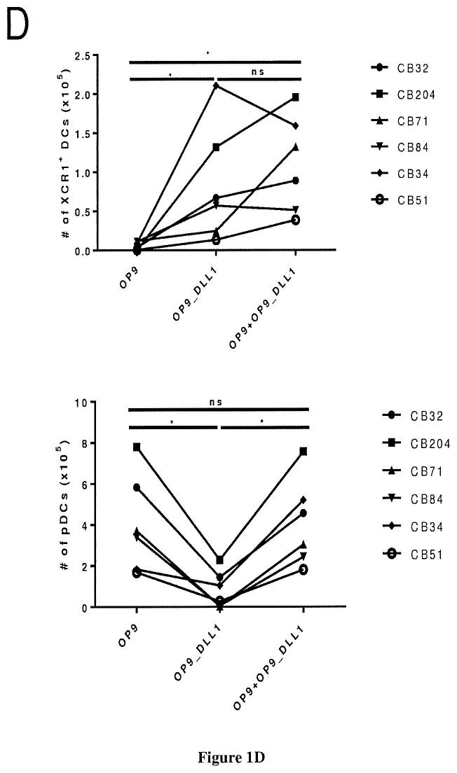

pDC can develop from human CD34⁺ progenitor cells isolated from cord blood (Olivier A, et al. The Notch ligand delta-1 is a hematopoietic development cofactor for plasmacytoid dendritic cells. Blood. 2006 Apr. 1; 107(7):2694-701), thymus or foetal liver (Dontje W, et al. Delta-like1-induced Notch1 signaling regulates the human plasmacytoid dendritic cell versus T-cell lineage decision through control of GATA-3 and Spi-B. Blood. 2006 Mar. 15; 107(6):2446-52) on OP9 stromal cells in the presence of FLT3-L and IL-7. However, opposite results were obtained between these two studies on the role of Notch1 signalling in the regulation of pDC development in this culture system. Moreover, the development of XCR1+ cDC in these cultures systems was not reported, and the role of Notch signalling on the differentiation of these cells is unknown. Thus, we investigated whether OP9 stromal cells would allow the simultaneous differentiation of both pDC and XCR1⁺ cDC from human CB CD34+ progenitors and how Notch signalling may affect this process (FIG. 1). CD34⁺ CB cells were first expanded in the presence of Flt3L, SCF, TPO, and IL7 (FST7) for 7 days. Expanded cells could then be either directly used for differentiation, transduced with lentiviral vectors prior to differentiation or frozen for later use. This expansion steps provides higher cell yields and increases assay flexibility. It simplifies screening different batches of CB CD34⁺ progenitors for their differentiation efficiency in order to choose the most suited one. It also enables using the same batch of amplified cells at different times, to use the same cell source to conduct complementary experiments or for successive rounds of vaccination. Expanded cells were differentiated on OP9, OP9_DL1, or OP9+OP9_DLL1 stromal cells for additional 14 to 21 days in the presence of Flt3L, TPO, and IL7 (FT7) (FIG. 1A). At the end of the culture, cells were harvested and stained with fluorescently labelled antibodies for analysis by flow cytometry. pDC were identified as CD123⁺BDCA2⁺ and XCR1⁺ DCs as BDCA3⁺CLEC9A⁺ (FIG. 1B). Similar to what was reported before when using thymus or foetal liver CD34⁺ progenitor cells cultured with FLT3-L and IL-7 (Dontje et al. Blood. 2006), OP9 cells allowed efficient generation of pDC. However, only a very low frequency of XCR1⁺ cDC differentiated under those experimental conditions (FIG. 1B, C). In contrast, in the presence of OP9_DLL1 a high frequency of XCR1⁺ cDC was found, but under these conditions pDC frequencies were lower than on OP9 cells not expressing DLL1 (FIG. 1B, C). Finally, differentiating the expanded CD34⁺ CB precursors on a mixed feeder layer combining OP9 and OP9_DLL1 allowed to reach maximal frequencies for both DC subsets within the same culture (FIG. 1B, C). From 10E4 expanded cells, on a mixed feeder layer of OP9+OP9_DLL1, the differentiation phase allowed to generate in average 1.1×10E5 XCR1⁺ cDC and 4.1×10E5 pDC (FIG. 1D). Prior to the differentiation phase, during the expansion phase, CD34⁺ CB cells multiply in average 2.9 fold. Thus, our culture system led to 3 to 20 times higher yields for XCR1⁺ cDC and pDC as compared to alternative methods (Table 1). In summary, the FT7 differentiation protocol allows for the simultaneous generation of uniquely large numbers of XCR1⁺ cDC and pDC. In addition, comparison of the frequencies and yields of pDC and XCR1⁺ cDC on stromal cells expressing or not DLL1 suggested that Notch signalling has opposite effects on the differentiation of these two cell types, inhibitory for the former but promoting for the later.

Role of Different Cytokines in the Promotion of the Differentiation of pDC and XCR1⁺ cDC on OP9 Feeder Layers.

Different concentrations and combinations of cytokines were tested during the differentiation phase to determine the combination the best suited to yield high numbers of both pDC and XCR1⁺ cDC in the same culture (data not shown). FLT3-L drove a better differentiation of both pDC and XCR1⁺ cDC at 15 ng/ml as compared to 5 ng/ml. Adding TPO to FLT3-L and IL-7 was not critical for the differentiation of these cell types but very significantly increased yields. Adding GM-CSF and IL-4 increased the frequency of XCR1⁺ cDC but at the expense of pDC. Adding IL-3, SCF or the aryl hydrocarbon receptor antagonist StemRegenin1 did not improve differentiation (data not shown). The replacement of the OP9 stromal cells by the MS5 ones led to much lower yields (data not shown). Hence, among all those we tested, the optimal culture conditions were those depicted above in the materials and methods section.

Kinetic Analysis of the Differentiation of pDC and XCR1⁺ cDC on OP9 Feeder Layers.

Expanded CD34⁺ cord blood cells were differentiated on OP-9, OP9_DLL1, or OP9+OP9_DLL1 feeder layer cells in the presence of FLT3-L, IL-7, and TPO for 14 to 28 days with medium changes every 7 days. The frequency of pDC and XCR1⁺ cDC was assessed at the initiation of the differentiation culture (d0) immediately after the expansion phase, as well as on days 14, 21 and 28 of differentiation. No pDC and only extremely low frequencies of XCR1⁺ cDC could be detected at d0 (data not shown). Much higher frequencies of these cells were observed at day 14 that further increased slightly at day 21, whereas cell numbers and DC frequencies had significantly decreased by d28 (data not shown). Hence, the numbers of pDC and XCR1⁺ cDC peak in the third week of differentiation.

Inhibition of Notch Signaling Blocks the Development of XCR1⁺ cDC In Vitro.

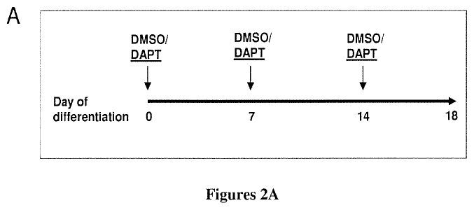

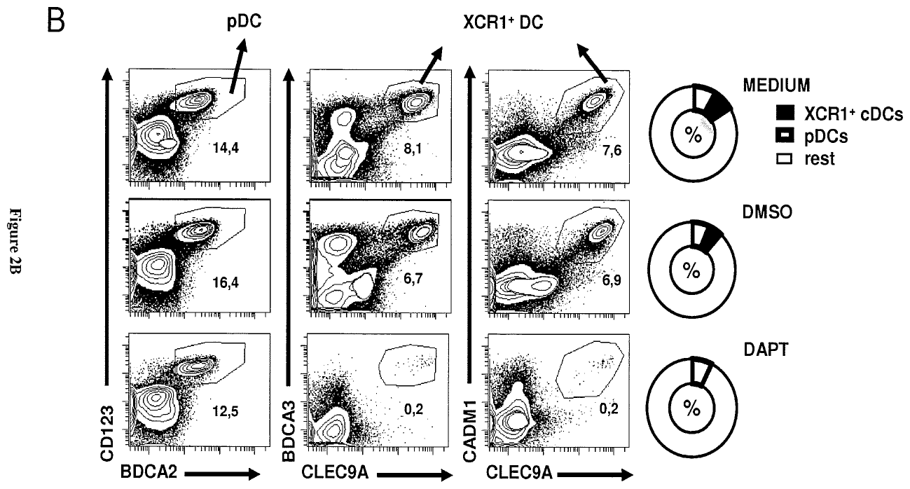

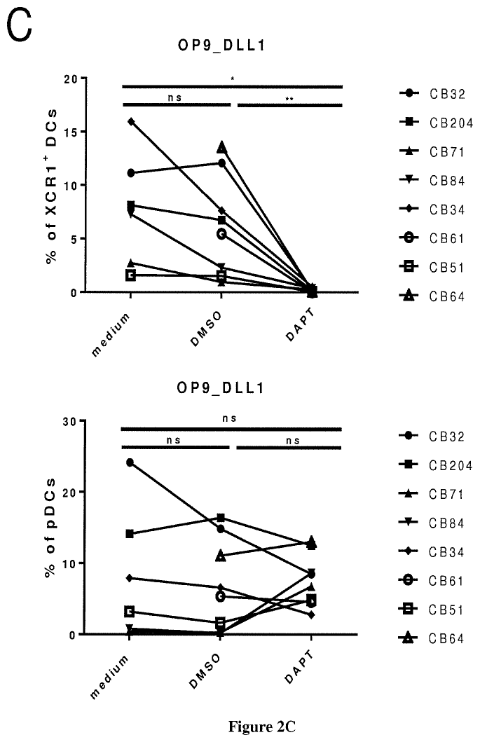

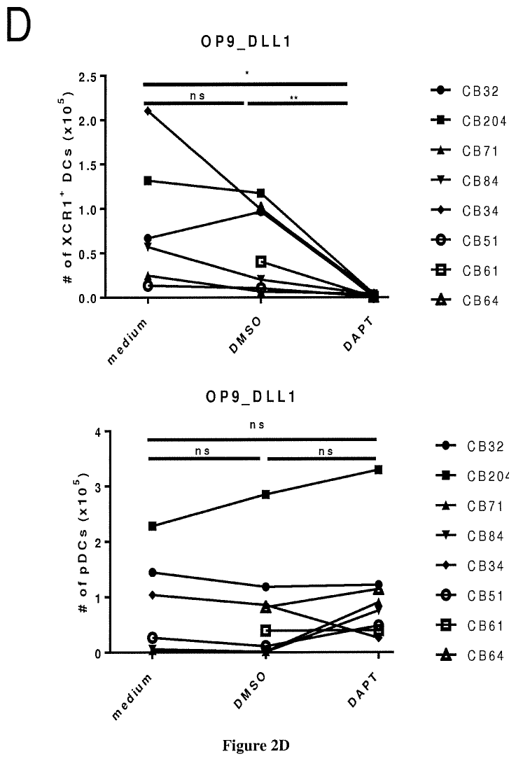

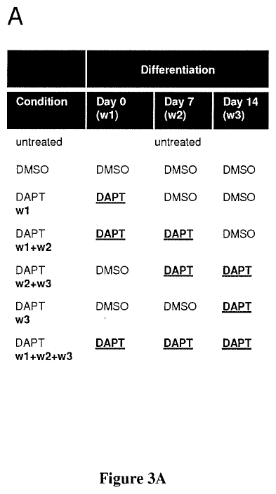

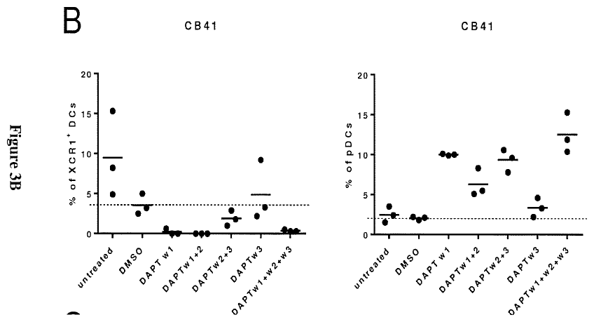

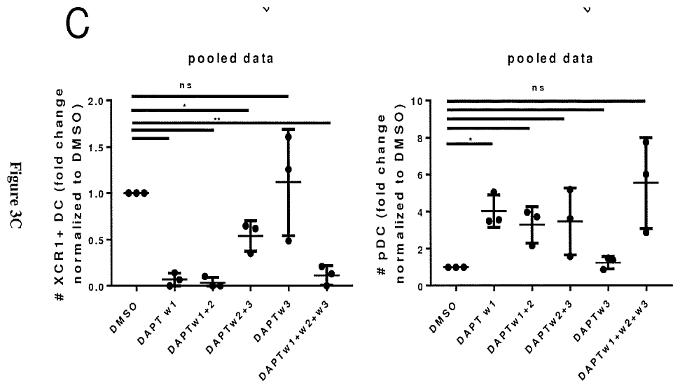

To evaluate in more detail the dependence of XCR1⁺ cDC on DLL1 and Notch-dependent downstream signalling for their differentiation, we tested whether we can block XCR1⁺ cDC development by using DAPT, an inhibitor of γ-secretase, which hinders Notch signalling. Indeed, when the FT7 cultures were treated with DAPT weekly during the whole period of differentiation (FIG. 2A), the frequency of XCR1⁺ cDC dropped dramatically, whereas the frequencies of pDC remained high as compared to the vehicle control (DMSO) (FIG. 2B, C). Similar results were observed for the total numbers of XCR1⁺ cDC and pDC in the cultures: Whereas pDC numbers were not affected by DAPT treatment, XCR1⁺ cDC numbers decreased significantly (FIG. 2D). In order to test at which phase during differentiation Notch signaling is required the most, we added DAPT at different time points (only in week 1, only in week 1+2, only in week 2+3, only in week 3), or throughout the whole period of differentiation (week 1+2+3) as before (FIG. 3A). We found that the inhibition of XCR1⁺ cDC development by DAPT is particularly strong when it is added at the beginning of differentiation (w1 or w1+2), whereas delayed addition had a much lower (w2+3) or even no (w3) impact (FIG. 3B, C). We conclude that DL1 and its downstream signalling is required for efficient in vitro differentiation of XCR1⁺ cDCs but dispensable for pDC development in our culture system. Furthermore, Notch signalling at early timepoints is required for efficient XCR1⁺ cDC in vitro differentiation.

In Vitro Generated pDCs and XCR1⁺ cDC Display Functional Characteristics of their In Vivo Equivalents.

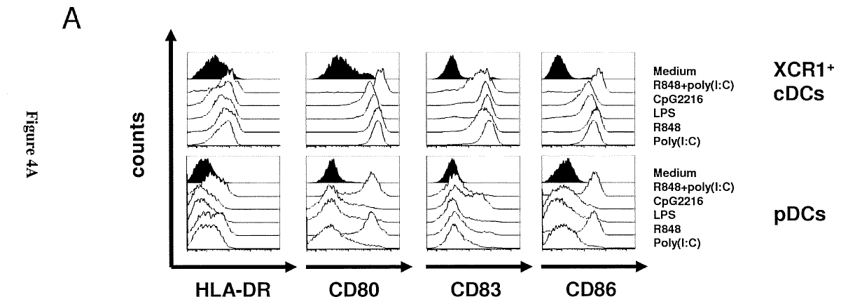

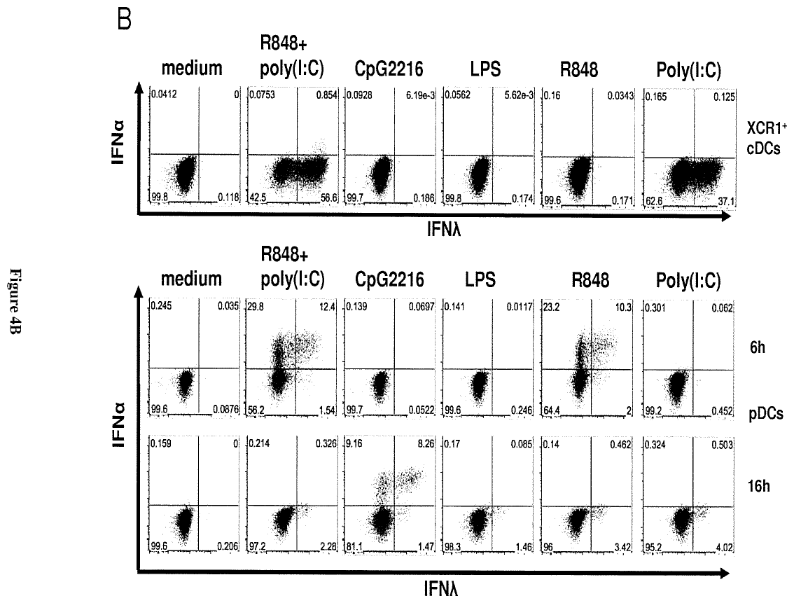

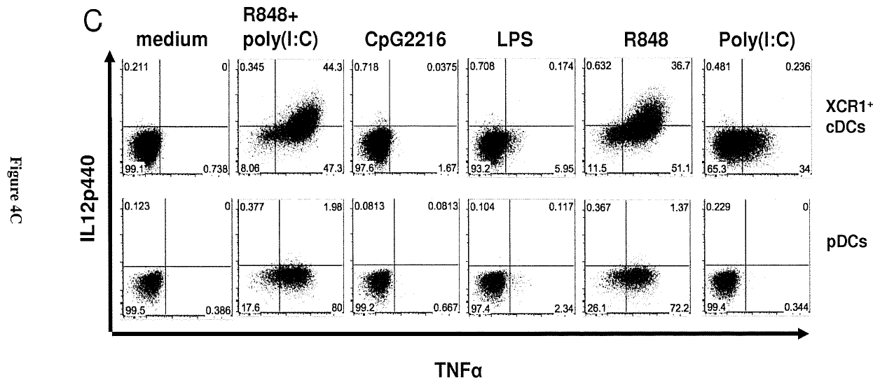

To examine whether in vitro generated pDC and XCR1⁺ cDC shared functional characteristics with their in vivo equivalents, we assessed their activation pattern and cytokine production upon stimulation with synthetic TLR ligands, at the single cell level, by flow cytometry. We used a panel of TLR agonists including R848 (TL7/8 agonist), poly(I:C) (TLR3 agonist), CpG2216 (TLR9 agonist), LPS (TLR4 agonist), and a combination of R848+poly(I:C). We observed that XCR1⁺ cDC upregulated HLA-DR as well as the activation markers CD80, CD83, and CD86 in response to all TLR agonists tested as compared to the medium control (FIG. 4A). By contrast, pDC mainly upregulated HLA-DR, CD80 and CD86 upon R848 or R848+poly(I:C) stimulation and CD83 only upon CpG2216 stimulation (FIG. 4A). A high proportion of in vitro derived XCR1⁺ cDC expressed IFN-λ but not IFN-α, only upon TLR3 triggering, i.e. stimulation with poly(I:C) or R848+poly(I:C) (FIG. 4B). They strongly expressed IL-12 only upon TLR8 triggering, i.e. stimulation with R848 or R848+poly(I:C) (FIG. 4C). TNF was induced in these cells both by TLR3 and TLR8 triggering (FIG. 4C). However, none of these cytokines were induced in XCR1⁺ cDC stimulated through TLR9 (CpG) or TLR4 (LPS). In contrast, pDC from the same cultures expressed cytokines only upon TLR7 (R848) or TLR9 (CpG) triggering, with a high induction of IFN-α and TNF, a milder expression of IFN-λ but not expression of IL-12 (FIG. 4B-C). Thus, the pDC and XCR1⁺ cDC generated in vitro in our culture system faithfully mirror the known TLR responses of their in vivo counterparts.

In Vitro Generated pDC and XCR1⁺ cDC Display Phenotypic Characteristics of their In Vivo Equivalents.

To better characterize our cultures, we analysed them for the surface expression of multiple classical DC subset markers. For a more unbiased analysis of our multi parameter flow cytometry data, we used the vi_SNE algorithm (Amir el-AD et al. viSNE enables visualization of high dimensional single-cell data and reveals phenotypic heterogeneity of leukemia. Nat Biotechnol. 2013 June; 31(6):545-52) which groups cell populations with similar expression patterns close to each other on the vi-SNE plots by taking into consideration all parameters analysed. When applied this algorithm to all live Lin⁻ HLA-DR⁺ cells (data not shown). We could thus identify a cluster of CD34(neg) CX3CR1(neg) BDCA2(low to neg) CD141(pos) CADM1(pos) CLEC9A(pos) BTLA(pos) cells, and a cluster of CD34(neg) CX3CR1(low to neg) CADM1(neg) CLEC9A(neg) XCR1(neg) CD1c(neg) CD11c(neg) CD123(pos) BDCA2(pos) LILRA4(pos) BTLA(pos) cells, matching the phenotypes of blood XCR1⁺ cDC and pDC respectively. Contrary to their blood counterparts, in vitro derived XCR1⁺ cDC also expressed CD1c. However, it has been reported previously that XCR1⁺ cDC derived in vitro from CB CD34⁺ progenitors on MS5 stromal cells or isolated from Flt3L-injected human volunteers upregulate their CD1c expression (Breton et al. J Exp. Med. 2015). CD1c expression could thus possibly be upregulated due to the high concentrations of Flt3L in our culture system. The cluster of in vitro derived XCR1⁺ cDC could be further divided into two subpopulations differing in their expression of CD123.

Single Cell RNA Sequencing Definitively Demonstrates the Homology Between In Vitro Derived XCR1⁺ cDC and pDC and their In Vivo Counterparts and Unravels an Overlooked Heterogeneity within XCR1⁺ cDC.

To further evaluate the degree of homology between the cells generated in vitro and their in vivo counterparts, and to assess possible heterogeneity of in vitro derived pDC and XCR1⁺ cDC, we performed single cell RNA sequencing from cells cultured on OP9+OP9_DLL1 under FT7 conditions. All cells were sorted from a live Lin(neg) HLA-DR(pos) gate. pDC were sorted as CD141(neg to low) CADM1(neg) BDCA2(pos) CD123(pos) cells. XCR1⁺ cDC were sorted as CD141(pos) CADM1(pos) cells. In addition, as external references, we included two other putative DC populations identified in the culture by multidimensional flow cytometry analyses using the vi_SNE algorithm: CD141(low to neg) CADM1(neg) BDCA2(neg) CD123(neg) CD1c(pos) BTLA(pos) cells versus CADM1(neg) BDCA2(neg) CD123(neg) CD1c(pos) BTLA(neg) cells. RNA isolation, downstream processing for sequencing and data bioinformatics analyses were performed based on a recently published method (Villani A C, et al. Single-cell RNA-seq reveals new types of human blood dendritic cells, monocytes, and progenitors. Science. 2017 Apr. 21; 356(6335)). An unsupervised t-SNE analysis of the data identified 7 clusters of cells, based only on their gene expression profiles (data not shown). One cluster contained only, and the immense majority of, sorted pDC. Only 2 out of the 15 cells sorted as putative pDC did not fall in this cluster. The genes identified as specifically expressed to high levels in this cluster as compared to all other clusters encompassed many genes known to be specific of pDC (Robbins S H, et al. Novel insights into the relationships between dendritic cell subsets in human and mouse revealed by genome-wide expression profiling. Genome Biol. 2008 Jan. 24; 9(1):R17) (Crozat K, et al. Comparative genomics as a tool to reveal functional equivalences between human and mouse dendritic cell subsets. Immunol Rev. 2010 March; 234(1):177-98), including GZMB, PTCRA, NLRP7, SPIB, LILRA4, PACSIN1, CLEC4C, LILRB4, TCF4, IL3RA, NRP1, IRF7, EPHA2, TLR7, TEX2, CXXC5, PLAC8 and BLNK. Moreover, for this cell cluster as compared to all other ones, GeneSet Enrichment Analyses (GSEA) identified the transcriptomic fingerprints previously established for pDC as the gene signatures the most significantly enriched (Robbins et al. Genome Biol. 2008); (Carpentier S, et al. Comparative genomics analysis of mononuclear phagocyte subsets confirms homology between lymphoid tissue-resident and dermal XCR1(+) DCs in mouse and, human and distinguishes them from Langerhans cells. J Immunol Methods. 2016, May; 432:35-49); (See P, et al. Mapping the human DC lineage through the integration of high-dimensional techniques. Science. 2017 Jun. 9; 356(6342)). Two clusters contained only, and all of the, cells sorted as putative XCR1⁺ cDC. The genes identified as specifically expressed to high levels in these clusters as compared to the other ones encompassed many genes known to be specific of XCR1⁺ cDC (Robbins et al. Genome Biol. 2008), including CADM1, CLEC9A, IDO1, C1orf54, BATF3, SLAMF8, SNX22, CPNE3, GCSAM, THBD, WDFY4, IDO2 and CLNK. Moreover, for these 2 cell clusters as compared to all other ones, GeneSet Enrichment Analyses (GSEA) identified the transcriptomic fingerprints previously established for XCR1⁺ cDC as the gene signatures the most significantly enriched (Robbins et al. Genome Biol. 2008; Carpentier et al. J Immunol Methods. 2016; Villani et al. Science. 2017; See et al. Science. 2017). Hence, Single cell RNA sequencing definitively demonstrated the homology between in vitro derived XCR1⁺ cDC or pDC and their in vivo counterparts. In addition, this approach unravelled an overlooked heterogeneity within XCR1⁺ cDC. Indeed, the two clusters identified for this cell type differed for the expression of cell cycle genes versus genes involved in the translation machinery and of CXCR4 versus XCR1. This suggested that our culture encompasses two differentiation states of XCR1⁺ cDC: terminally differentiated cells expressing XCR1 versus their immediate precursors negative for XCR1 but expressing higher levels of CXCR4 and of cell cycle genes, which had not been identified before to the best of our knowledge. Flow cytometry analysis of in vitro derived CLEC9A⁺CADM1⁺ cDC confirmed that these cells encompass two complementary populations based on their expression of XCR1 and CXCR4, and that this is also the case for their blood counterpart (data not shown).

TABLE 1

| | Cord blood sample identity | | | | | | | |
|---|---|---|---|---|---|---|---|---|
| Feeder layer | CB32 | CB204 | CB71 | CB84 | CB34 | CB51 | mean | SD |

Total fold increase of live cells

| | | | | | | | | | |
|---|---|---|---|---|---|---|---|---|---|
| Expansion¹ | | 2.67 | 3.2 | 2.13 | 2.46 | 1.5 | 5.6 | 2.93 | 1.43 |
| Expansion & | OP9 | 641 | 870 | 682 | 541 | 216 | 829 | 630 | 236 |
| differentiation² | OP9_DL1 | 160 | 518 | 192 | 192 | 198 | 470 | 288 | 161 |
| | OP9 + OP9_DL1 | 363 | 960 | 328 | 472 | 360 | 504 | 498 | 237 |

Total numbers of XCR1+ cDC (×10E5) generated from 10E4 human CD34+ cord blood cells.³

| | | | | | | | | |
|---|---|---|---|---|---|---|---|---|
| OP9 | 0.10 | 0.00 | 0.26 | 0.27 | 0.14 | 0.04 | 0.14 | 0.11 |
| OP9_DL1 | 1.79 | 4.21 | 0.53 | 1.40 | 3.16 | 0.75 | 1.97 | 1.44 |
| OP9 + OP9_DL1 | 2.37 | 6.25 | 2.81 | 1.26 | 2.38 | 2.16 | 2.87⁴ | 1.73 |

Total numbers of pDC (×10E5) generated from 10E4 human CD34+ cord blood cells.³

| | | | | | | | | |
|---|---|---|---|---|---|---|---|---|
| OP9 | 15.57 | 24.98 | 7.89 | 8.35 | 2.75 | 9.45 | 11.50 | 7.77 |
| OP9_DL1 | 3.87 | 7.31 | 0.08 | 0.16 | 1.56 | 1.51 | 2.42 | 2.76 |
| OP9 + OP9_DL1 | 12.18 | 24.19 | 6.46 | 6.00 | 7.81 | 10.17 | 11.14⁵ | 6.81 |

¹Calculations are based on the expansion of 5,000 CD34⁺ CB cells/well under FST7 conditions.
²Calculations are based on the expansion of 5,000 CD34⁺ CB cells/well under FST7 conditions with subsequent differentiation of 10,000 expanded cells/well under FT7 conditions on the indicated feeder layers for 18-19 days.
³Calculations are based on the expansion of 5,000 CD34⁺ CB cells/well under FST7 conditions with subsequent differentiation of 10,000 expanded cells/well under FT7 conditions on the indicated feeder layers for 18-19 days. XCR1+ cDC and pDC were gated as described in FIG. 1B.
⁴For comparison, equivalent yields were 1.2 for CD141(pos)CLEC9A(neg-to-pos) cells and thus less than that for bona fide CD141(pos)CLEC9A(pos) cells in (Thordardottir et al. Stem cells and development. 2014) and 0.25 in (Lee et al. J Exp Med. 2015), thus about 3 to 10 times less than with our protocol.
⁵For comparison, equivalent yields were 3.8 in (Thordardottir et al. Stem cells and development. 2014) and 0.5 in (Lee et al. J Exp Med. 2015), thus about 3 to 20 times less than with our protocol.

### REFERENCES

Throughout this application, various references describe the state of the art to which this invention pertains. The disclosures of these references are hereby incorporated by reference into the present disclosure.

1. A method of obtaining a mixed population of human XCR1⁺ and plasmacytoid dendritic cells (DC) said method comprising the steps of i) culturing, in a culture medium, a population of human hematopoietic stem cells (HSC) or more committed hematopoietic precursor cells in the presence of a Notch ligand, and thereafter, ii) isolating human XCR1⁺ and plasmacytoid DC from the culture.

2. The method of claim 1 wherein the population of human hematopoietic stem cells is a population of CD34⁺ cells that have been isolated, or partially purified, from cord blood.

3. The method of claim 1 wherein the Notch ligand is Delta1 (Delta-like 1/DLL1), or Delta4 (Delta-like 4/DLL4).

4. The method of claim 1 wherein the Notch ligand is immobilized on a solid phase.

5. The method of claim 1 wherein the Notch ligand is provided to the culture medium by the inclusion of suitable feeder cells.

6. The method of claim 5 wherein the feeder cells are OP9-DLL1 feeder cells.

7. The method of claim 1 wherein the human hematopoietic stem cells are co-cultured with a mixture of feeder cell that express the Notch ligand and feeder cells that do not express the Notch ligand.

8. The method of claim 7 wherein the human hematopoietic stem cells are co-cultured with a mixture of OP9 and OP9-DLL1 cells.

9. The method of claim 1 wherein the culture medium comprises an amount of at least one human cytokine that is suitable for enhancing the DC differentiation or expansion that occurs during the step of culturing to thereby increase the relative amount of XCR1⁺ DC.

10. The method of claim 9 wherein the at least one human cytokine is selected from the group consisting of Fms-like tyrosine kinase 3 ligand (FLT3-L), interleukin 7 (IL-7) and thrombopoietin (TPO).

11. The method of claim 1 wherein the culture medium comprises an amount of FLT3-L, IL-7 and TPO.

12. The method of claim 1 wherein the duration of the culturing step is in the range of about 5 to 25 days.

13. The method of claim 14 wherein the duration of the culturing step is 14, 15, 16, 17, 18, 19, 20 or 21 days.

14. A method for the preparation of a DC vaccine comprising
obtaining a mixed population of human XCR1⁺ and plasmacytoid dendritic cells (DC) by the method of claim 1,
isolating plasmacytoid DC from the culture, and
preparing a vaccine comprising a therapeutically effective amount of the plasmacytoid DC.

15. The method of claim 4 wherein the solid phase is the surface of a tissue culture dish, a flask, or a bead.

16. The method of claim 12, wherein the duration of the culturing step is in the range of about 14 to 21 days.
